(19) United States
(12) Patent Application Publication
(10) Pub. No.: US 2024/0248082 A1
(43) Pub. Date: Jul. 25, 2024

(54) MARKER, METHOD AND DEVICE FOR ANALYZING A BIOLOGICAL SAMPLE

(71) Applicant: LEICA MICROSYSTEMS CMS GMBH, Wetzlar (DE)

(72) Inventor: Soeren ALSHEIMER, Wetzlar (DE)

(21) Appl. No.: 18/290,463

(22) PCT Filed: Dec. 23, 2021
(86) PCT No.: PCT/EP2021/087551

§ 371 Date: Nov. 14, 2023

(30) Foreign Application Priority Data
May 19, 2021 (WO) PCT/EP2021/063310
Jun. 18, 2021 (WO) PCT/EP2021/066645
Aug. 28, 2021 (WO) PCT/EP2021/073819

(51) Int. Cl. G01N 33/533; G01N 21/64

(52) U.S. Cl. CPC G01N 33/533; G01N 21/6408; G01N 21/6428; G01N 21/6458; G01N 2021/6419; G01N 2021/6421; G01N 2021/6441

(57) ABSTRACT

A marker for marking a predetermined structure within a biological sample includes an affinity reagent configured to attach to the predetermined structure, a linker structure attached to the affinity reagent and extending from the affinity reagent, and at least two different fluorescent dyes arranged at the linker structure. The linker structure includes at least one cleavage site arranged between the two fluorescent dyes or between one of the fluorescent dyes and the linker structure. The linker structure is capable of being cut at the cleavage site by a cleaving agent in order to remove at least one of the fluorescent dyes from the marker.

### CROSS REFERENCE TO RELATED APPLICATIONS

This application is a U.S. National Phase application under 35 U.S.C. § 371 of International Application No. PCT/EP2021/087551, filed on Dec. 23, 2021, and claims benefit to International Application No. PCT/EP2021/073819, filed on Aug. 28, 2021 and International Application No. PCT/EP2021/066645, filed on Jun. 18, 2021 and International Application No. PCT/EP2021/063310, filed on May 19, 2021. The International Application No. PCT/EP2021/087551 was published in English on Nov. 24, 2022 as WO 2022/242895 A1 under PCT Article 21(2).

### FIELD

Embodiments of the present invention relate to marker for marking a predetermined structure within a biological sample. Embodiments of the present invention also relate to a method for analyzing a biological sample and a device for analyzing a biological sample.

### BACKGROUND

In order to address key problems in the field of life sciences it is vital to precisely identify and to locate certain structures within biological samples, e.g. tissue samples or cell cultures. This can be done by introducing markers into the sample that bind to specific structures, e.g. specific biomolecules. These markers typically comprise an affinity reagent that attaches to the structure in question and one or more fluorescent dyes that are either directly conjugated to the affinity reagent or attached to the affinity reagent by other means, for example a secondary affinity reagent.

Fluorescence microscopy for example allows for imaging the sample with high spatial resolution but involves only a low number of different fluorescent dyes, typically between 1 and 5. The available dyes have to be distributed to all markers that are used to identify cell types, functional markers like protein-of-interest and general morphological markers in the same experiment. This means that cell types in most imaging experiments are merely poorly identified. While modern approaches that allow for a much more reliable and robust identification of cell types, e.g. based on the analysis of genetic regulatory networks (GRNs), exist they require a much higher number of different markers to be read-out from the sample.

The documents PCT/EP2021/063310 and PCT/EP2021/073819 propose markers and methods each for increasing the number of markers that can be used in a single fluorescence microscopy experiment. Each marker comprises a unique combination of dyes forming a code, that in principle identifies the respective marker. However, certain ambiguities remain, in particular when a large number of markers is in close proximity of each other. In order to resolve these ambiguities, it is necessary to remove the markers and repeat the image acquisition with a different set of markers. This process is time consuming and expensive.

### SUMMARY

Embodiments of the present invention provide a marker for marking a predetermined structure within a biological sample. The marker includes an affinity reagent configured to attach to the predetermined structure, a linker structure attached to the affinity reagent and extending from the affinity reagent, and at least two different fluorescent dyes arranged at the linker structure. The linker structure includes at least one cleavage site arranged between the two fluorescent dyes or between one of the fluorescent dyes and the linker structure. The linker structure is capable of being cut at the cleavage site by a cleaving agent in order to remove at least one of the fluorescent dyes from the marker.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view of a marker for marking a predetermined structure within a biological sample according to an embodiment;

FIG. 2 is a schematic view of the marker according to FIG. 1 after the introduction of a first cleaving agent according to an embodiment;

FIG. 3 is a schematic view of the marker according to FIGS. 1 and 2 after the removal of a first fluorescent dye according to an embodiment;

FIG. 4 is a schematic view of the marker according to FIGS. 1 to 3 after the introduction of a second cleaving agent according to an embodiment;

FIG. 5 is a schematic view of the marker according to FIGS. 1 to 5 the removal of the first fluorescent dye and a second fluorescent dye according to an embodiment;

FIG. 6 is a schematic view of the marker according another embodiment;

FIG. 7 is a flowchart of the method for analyzing the biological sample utilizing the marker described above with reference to FIGS. 1 to 6 according to an embodiment;

FIG. 8 shows a schematic drawing of the arrangements of the fluorescent dyes of two different markers according to an embodiment; and

FIG. 9 shows a schematic drawing of a device for analyzing a biological sample according to an embodiment.

### DETAILED DESCRIPTION

Embodiments of the present invention provide a marker for marking a predetermined structure within a biological sample, a method for analyzing a biological sample and a device for analyzing a biological sample that allows for the reliable identification of a high number of predetermined structure at a low cost and time expenditure.

According to some embodiments, the marker for marking a predetermined structure within a biological sample comprises an affinity reagent configured to attach to the predetermined structure, a linker structure attached to the affinity reagent and extending from the affinity reagent, and at least two different fluorescent dyes arranged at the linker structure. The linker structure comprises at least one cleavage site arranged between the two fluorescent dyes or between one of the fluorescent dyes and the linker structure. The linker structure can be cut at the cleavage site by a cleaving agent in order to remove at least one of the fluorescent dyes from the marker.

The affinity reagent may in particular be an antibody, a single-domain antibody (also known as nanobody), a combination of at least two single-domain antibodies, an aptamer, an oligonucleotide, a morpholino, a PNA complementary to a predetermined RNA, DNA target sequence, a ligand (for example a drug or a drug-like molecule), or a toxin, for example a Phalloidin a toxin that binds to an actin filament. An affinity reagent in the sense of this document may be homo-multimer or hetero-multimer of affinity reagents, which provides the advantage of introducing an avidity effect, which effectively leads to higher sensitivity in the detection of analytes. The predetermined structure may be a specific bio-molecule, for example a protein, an RNA sequence, a peptide, a DNA sequence, a metabolite, a hormone, a neurotransmitter, a vitamin or a micronutrient. The predetermined structure may also by a single analyte, for example a metal ion, in particular a heavy metal ion such as Cd(II), Co(II), Pb(II), Hg(II) or U(VI).

In the sense of this document “plurality of affinity reagents” (S₂) contains the affinity reagents (a₁, a₂, a₃, . . . aₙ), which are configured to specifically bind to a predetermined target structure within the biological sample or to a predetermined chemical compound or to a predetermined chemical element. At least some of the affinity reagents from the plurality of affinity (A) reagents are “introduced to the sample” such that the affinity reagents can attach to the respective predetermined target structure within the sample. In this context and in the sense of this document and as described above “introduced to the sample” may refer to being physically introduced into the volume of the sample or into a volume surrounding and assigned to the sample. An example of the latter case may be assays for secreted molecules for instance, which are best assessed in the extracellular space where they might be outside of the sample, but within a certain spatial context or vicinity.

The affinity reagent attaches itself to the predetermined structure when it is introduced into the sample. Thereby, the different fluorescent dyes are attached to the predetermined structure making the predetermined structure visible to fluorescence imaging. Each marker comprises a unique arrangement of fluorescent dyes forming a code that in principle uniquely identifies the respective marker. In order to decode the arrangement, the linker structure is cut at the cleavage site in successive round or cycles of imaging. By cutting the linker structure at the cleavage site, one of the fluorescent dyes is removed from the marker. This means that the removed fluorescent dye can be washed out of the sample. By removing a fluorescent dye, the arrangement of the fluorescent dyes on the linker structure, and thus the code represented by the arrangement is changed. This code change can be used to resolve ambiguities that arise in a first round of imaging in later rounds of marking without necessitating the removal or reintroduction of affinity reagents. This in turn reduces cost and time expenditure for experiments involving a high number markers, i.e. a high number of predetermined structures to be identified.

The linker structure provides support for the arrangement of fluorescent dyes. The arrangement may in particular be a linear arrangement in which the fluorescent dyes are arranged in a particular order along the linker structure extending from the affinity reagent.

“Linker”: In the sense of this document the linker denotes a unipartite chemical structure (e.g. a monomeric molecule or a polymer) or multipartite assembly of chemical structures linking a combination of fluorescent dyes to an affinity reagent. A linker might be directly or covalently coupled to the dyes and to the affinity reagent or indirectly through for example affinity tag-affinity ligand combination such as streptavidin-biotin interaction or a hapten or an oligonucleotide for example. In the case of covalent coupling this may be a site-selective coupling. Commonly used coupling chemistries such NHS-, maleimide, azide-alkine and a range of further so called click chemistries may be used to couple the linker to the affinity reagent and/or the linker to a dye. A linker may in particular comprise an oligonucleotide (e.g. DNA, RNA, LNA, PNA, morpholino, other artificial oligonucleotide), a peptide, a DNA-origami-based structure such as for example a nanoruler, a micro-/nanobead, a polymer, a micro-/nanocapsule, a micro-/nanocrystal, a carbontube, a carbon-based nanostructure (e.g. a graphene). A linker may in particular comprise an oligonucleotide and another element of the group mentioned before, like for example comprise an oligonucleotide and a peptide.

“Sample”: In the sense of this document “sample” refers to a “biological sample” which may also be named a “biological specimen” or “specimen” including, for example blood, serum, plasma, tissue, bodily fluids (e.g. lymph, saliva, semen, interstitial fluid, cerebrospinal fluid), feces, solid biopsy, liquid biopsy, explants, whole embryos (e.g. zebrafish, Drosophila), entire model organisms (e.g. zebrafish larvae, Drosophila embryos, C. elegans), cells (e.g. prokaryotes, eukaryotes, archea), multicellular organisms (e.g. Volvox), suspension cell cultures, monolayer cell cultures, 3D cell cultures (e.g. spheroids, tumoroids, organoids derived from various organs such as intestine, brain, heart, liver, etc.), a lysate of any of the aforementioned, a virus. In the sense of this document “sample” further refers to a volume surrounding a biological sample, like for example in assays, where secreted proteins like growth factors, extracellular matrix constituents are being studies the extracellular environment surrounding a cell up to a certain assay-dependent distance, is also referred to as the “sample”. Specifically, affinity reagents brought into this surrounding volume are referred to in the sense of this document as being “introduced into the sample”. In the sense of this document a “sample” may be used for uses in at least one of the following areas basic, applied or translational research, diagnostic procedures, drug discovery and development, biotechnology and bioprocessing applications, environmental sciences and environmental protection, quality control and quality assurance.

“Analyte”, “Predetermined target structure”, “Target”: In the sense of this document “predetermined target structure”, “analyte”, and “target” are used synonymously and refer to a target element, target molecule or a target structure, which may for example be a protein (e.g. a certain protein), an RNA sequence (e.g. the mRNA of a certain gene), a peptide (e.g. somatostatin), a DNA sequence (e.g. the a genetic locus or element), a metabolite (e.g. lactic acid), a hormone (e.g. estradiol), a neurotransmitter (e.g. dopamine), a vitamin (e.g. cobalamine), a micronutrient (e.g. biotin), a metal ion (e.g. metal and heavy metal ions like Cd(II), Co(II), Pb(II), Hg(II), U(VI)). Analytes like for example proteins may assayed to measure the level of expression of proteins of interest, similar mRNA targets may be measured to assess gene expression levels of the respective genes from which the respective mRNA species are being transcribed.

“Dye”: In the sense of this document the terms “fluorescent dye”, “fluorophore”, “fluorochrome”, “dye” are used interchangeably to denote a fluorescent chemical compound or structure and can be in particular one of the following: a fluorescent organic dye, a fluorescent quantum dot, a fluorescent dyad, a fluorescent carbon dot, graphene quantum dot or other carbon-based fluorescent nanostructure, a fluorescent protein, a fluorescent DNA origami-based nanostructure. From the organic fluorescent dyes in particular derivatives of the following are meant by the term “fluorescent dye”: xanthene (e.g. fluorescein, rhodamine, Oregon green, Texas), cyanine (e.g. cyanine, indocarbocyanine, oxacarbocyanine, thiacarbocyanine, merocyanine), derivatives, squarine rotaxane derivatives, naphthalene, coumarin, oxadiazole, anthracene (anthraquinones, DRAQ5, DRAQ7, CyTRAK Orange), pyrene (cascade blue), oxazine (Nile red, Nile blue, cresyl violet, oxazine 170), acridine (proflavine, acridine orange, acridine yellow), arylmethine (auramine, crystal violet, malachite green), tetrapyrrole (porphin, phthalocyanine, bilirubin), dipyrromethene (BODIPY, aza-BODIPY). The following trademark groups designated commercially available fluorescent dyes, which may include dyes belonging to different chemical families CF dye (Biotium), DRAQ and CyTRAK probes (BioStatus), BODIPY (Invitrogen), EverFluor (Setareh Biotech), Alexa Fluor (Invitrogen), Bella Fluore (Setareh Biotech), DyLight Fluor (Thermo Scientific), Atto and Tracy (Sigma-Aldrich), FluoProbes (Interchim), Abberior Dyes (Abberior Dyes), Dy and MegaStokes Dyes (Dyomics), Sulfo Cy dyes (Cyandye), HiLyte Fluor (AnaSpec), Seta, SeTau and Square Dyes (SETA BioMedicals), Quasar and Cal Fluor dyes (Biosearch Technologies), SureLight Dyes (Columbia Biosciences), Vio Dyes (Milteny Biotec) [list modified from: https://en.wikipedia.org/wiki/Fluorophore]. From the group of fluorescent proteins in particular the members of the green fluorescent protein (GFP) family including GFP and GFP-like proteins (e.g DsRed, TagRFP) and their (monomerized) derivatives (e.g., EBFP, ECFP, EYFP, Cerulaen, mTurquoise2, YFP, EYFP, mCitrine, Venus, YPet, Superfolder GFP, mCherry, mPlum) are meant by the term “fluorescent dye” in the sense of this document. Further from the group of fluorescent proteins the term “fluorescent dye” in the sense of this document may include fluorescent proteins, whose absorbance or emission characteristics change upon binding of ligand like for example BFPmsl or in response to changes in the environment like for example redox-sensitive roGFP or pH-sensitive variants. Further from the group of fluorescent proteins the term “fluorescent dye” in the sense of this document may include derivative of cyanobacterial phycobiliprotein small ultra red fluorescent protein smURFP as well as fluorescent protein nanoparticles that can be derived from smURFP. An overview of fluorescent proteins can be found in Rodriguez et al. 2017 in Trends Biochem Sci. 2017 February; 42(2): 111-129. The term “fluorescent dye” in the sense of this document may further refer to a fluorescent quantum dot. The term “fluorescent dye” in the sense of this document may further refer to fluorescent carbon dot, a fluorescent graphene quantum dot, a fluorescent carbon-based nanostructure as described in Yan et al. 2019 in Microchimica Acta (2019) 186: 583 and Iravani and Varma 2020 in Environ Chem Lett. 2020 Mar. 10: 1-25. The term “fluorescent dye” in the sense of this document may further refer to a fluorescent polymer dot (Pdot) or nanodiamond. The term “fluorescent dye” in the sense of this document may further refer to a fluorescent dyad, like for example a dyad of a perylene antenna and a triangelium emitter as described in Kacenauskaite et al. 2021 J. Am. Chem. Soc. 2021, 143, 1377-1385. The term “fluorescent dye” in the sense of this document may further refer to an organic dye, a dyad, a quantum dot, a polymer dot, a graphene dot, a carbon-based nanostructure, a DNA origami-based nanostructure, a nanoruler, a polymer bead with incorporated dyes, a fluorescent protein, an inorganic fluorescent dye, a SMILE, or a microcapsule filled with any of the aforementioned. The term “fluorescent dye” in the sense of this document may further refer to a FRET-pair having at least one fluorescent dye as FRET donor and at least one fluorescent dyes as a FRET acceptor, or a FRET-triple, which is used to generate a three component Forster resonance energy transfer. In particular, the FRET-pair or -triplet is connected by complementary linker. The term “fluorescent dye” in the sense of this document may further refer to a FRET n-tupel of physically connected dyes.

In a preferred embodiment, the affinity reagent comprises an attachment site and the linker structure comprises a complementary attachment site configured to attach to the attachment structure of the affinity reagent. In this embodiment the linker structure and the affinity reagent are provided separately. This can be used to provide the affinity reagent for a specific application while using generic linker structures. Thereby, the versatility and cost-effectiveness of the marker is further enhanced.

In another preferred embodiment, the linker structure is formed by oligonucleotides or peptides. The linker structure holds the fluorescent dyes in their arrangement. The fluorescent dyes may be directly, i.e. covalently, or indirectly, for example by an affinity tag-affinity ligand combination, coupled to the linker structure. The linker structure itself may comprise one or more oligonucleotides, for example DNA, RNA, LNA, PNA, a morpholino or another artificial oligonucleotide. The linker structure may also comprise peptides or other DNA- or RNA-analogues. Oligonucleotides and peptides alike can be synthesized to suit specific needs by standardized methods. The reporters can thus be produced easily and cost-effectively.

In another preferred embodiment, the cleavage site is an enzymatic cleavage site and the attachment structure can be cut at the cleavage site by an enzymatic cleaving agent. Preferably, the enzymatic cleavage site is a target site of a restriction enzyme, a CRISPR/Cas target, a recombinase target site, in particular a loxP site or a flippase target site, or a caspase target site. In order to remove one or more of the fluorescent dyes from the linker structure, the enzymatic cleaving agent is introduced into the sample. After the enzymatic cleaving agent has cut the linker structure at the cleavage site, the cut of part of the linker structure and the fluorescent dyes connected to this part of the linker structure can be washed out of the sample. Enzymatic cleaving has the advantage of targeting only the cleavage site, thereby reducing damage to other structures.

In another preferred embodiment, the cleavage site is a photocleavage site and the linker structure can be cut at the cleavage site by photolysis, in particular by UV light. Cutting the linker structure by means of photolysis is very efficient and easy to automate.

In a further preferred embodiment, the cleavage site is a proteolytic cleavage site and the linker structure can be cut at the cleavage site by a protease, in particular TEV protease or Factor Xa.

Embodiments of the present invention also relate to a method for analyzing a (biological) sample. The method comprises the following steps: a) Providing at least two markers according to any one of the claims 1 to 6, wherein the arrangement of the fluorescent dyes or the cleavage site at the linker structure is unique for each of the markers. b) Introducing the markers into the sample. c) Directing at least one first excitation light onto the sample in order to excite fluorescent dyes of the markers. d) Generating at least one first readout from fluorescence light emitted by excited fluorescent dyes located in a readout volume of the sample. e) Introducing at least one cleaving agent into the sample in order to remove at least one fluorescent dye from a respective marker. f) Directing at least one second excitation light onto the sample in order to excite remaining fluorescent dyes of the markers. g) Generating at least one second readout from fluorescence light emitted by excited remaining fluorescent dyes located in a readout volume of the sample. h) Determining the markers present in the readout volume based on the first and second readouts.

Optionally steps e) to g) may be carried out at least one more time before the markers present in the readout volume based are determined in step h).

The method uses one or more markers as described above in order to analyze the sample. The markers are used to target different predetermined structures in the sample in order to make them visible and identifiable. Individual markers are identified by their unique arrangement of fluorescent dyes. In order to uniquely identify the arrangement, the first and second readouts are used which comprise information about the emitted fluorescence light, in particular an emission spectrum, a fluorescence emission intensity or a fluorescence lifetime of the fluorescent dyes. Since the arrangement is changed by removing one of the fluorescent dyes between the capture of the first and second readouts, ambiguities arising during the capture of the first readout can be resolved in subsequent readouts. This allows for the reliable identification and separation of a high number of markers, and thus a high number of predetermined structures to be identified.

In another preferred embodiment, generating the first and/or second readouts comprises separating the emission light emitted by the excited fluorescent dyes into detection channels. The detection channels correspond to at least one emission characteristic of the fluorescent dyes. The emission characteristic is one of the following: an emission spectrum, a fluorescence intensity, a fluorescence lifetime, and an excitation fingerprint. Preferably, the marker is provided such that each fluorescent dye of the first and second reporter corresponds to one detection channel of the first and second readout, respectively. The channels are generated in order to separate the contributions of the different fluorescent dyes to a single readout. Generating the channels may comprise at least one of spectral unmixing, a determination of a fluorescence lifetime and determination of an excitation fingerprint of the fluorescent dyes. Spectral unmixing (also referred to as spectral imaging and linear unmixing, or channel unmixing) may be performed in various ways including but not limited to linear unmixing, principle component analysis, learning unsupervised means of spectra, support vector machines, neural networks, (spectral) phasor approach, and Monte Carlo unmixing algorithm. In order to reduce crosstalk between the fluorescent dyes associated with different markers, several of these techniques may be employed. The unmixing techniques can also be used to separate contributions from different fluorescent dyes, i.e. the crosstalk due to overlapping emission spectra, in for example a single pixel of a readout. Employing these techniques can greatly enhance the sensitivity of the method due to reduced noise. Further, the fluorescence lifetime and the excitation fingerprint of a fluorescent dye can be used in order to correctly identify fluorescent dyes, and thus reporters. This can be used to employ more sets of markers per readout. In turn, this vastly increases the overall number of markers that can be used in a single experiment.

In another preferred embodiment, all fluorescent dyes of the reporters are divided into sets of dyes. Each fluorescent dye in the same set can be excited by essentially one wavelength spectrum or by the same wavelength spectrum. In steps c) and f) at least one excitation light for each set of dyes is directed at the sample in order to excite the fluorescent dyes of the respective set. In steps d) and g) at least one readout for each set of dyes is generated from fluorescence light emitted by the excited fluorescent dyes located in the readout volume of the sample. Each set of fluorescent dyes may be excited independently by a different excitation light and, thus, fluorescence light emitted by each set may also be detected independently. This is used to generate multiple readouts, each capturing fluorescence light emitted by a different set. In each readout different fluorescent dyes are excited allowing a more robust identification of the different dyes, and thus the markers and associated predetermined structures of the sample.

In another preferred embodiment, in steps c) and f) the excitation lights are directed onto the sample in a sequence temporally following each other. Preferably, the time between applying the different excitation lights is longer than the fluorescence lifetime of the fluorescent dyes. Thereby, crosstalk between different fluorescent dyes can be reduced and the sensitivity of the method is further improved.

In another preferred embodiment, the cleaved fluorescent dye is washed out of the sample before the second excitation light is directed onto the sample. Washing out unbound fluorescent dyes ensures that only fluorescent dyes that are actually attached to their associated markers are detected. Thus, washing out unbound fluorescent dyes prevents misidentification of structures in the sample and makes the method more reliable.

In another preferred embodiment, the first and/or second readout comprises at least one image of the readout volume or a readout image data stream of the readout volume. In particular, the method comprises the further step of capturing a hyperspectral image of the sample in order to generate the first and/or second readouts. In contrast to multispectral imaging, which captures a limited number of wavelength bands, typically less than or around 10, a hyperspectral image captures tens or hundreds of wavelength bands per pixel. In other words, hyperspectral images have a very high spectral resolution. This allows for a much finer differentiation of fluorescent dyes based on their emission spectrum and thereby increases the sensitivity and reliability of the method.

In another preferred embodiment, the method comprises the further step of stabilizing the fluorescence lifetime of at least one fluorescent dye, in particular by placing the fluorescent dye in a shielded environment by at least one of encapsulating, polymer-matrix embedding, and co-crystallizing. Stabilizing the fluorescence lifetime allows for the much more reliable identification of the stabilized fluorescent dyes based on their lifetime. The method can also be used to increase the fluorescence lifetime of some fluorescent dyes of otherwise equal fluorescent dyes, providing a further differentiating feature, and thereby increasing the number of fluorescent dyes that can be used in a single experiment.

Embodiments of the present invention further relate to a device for analyzing a biological sample. The device is adapted to carry out the method described above.

The device has the same advantages as the method described above and can be supplemented using the features of the dependent claims directed at the method.

In a preferred embodiment, the device comprises a microscope, a plate reader, a cytometer, an imaging cytometer, or a fluorescence activated cell sorter configured to generate the first and second readouts. The microscope is preferably a lens-free microscope, a light field microscope, a widefield microscope, a fluorescence widefield microscope, a light sheet microscope, a scanning microscope, a spinning disc microscope or a confocal scanning microscope.

In another preferred embodiment, the device is configured to determine at least one of the following: a fluorescence emission intensity, a fluorescence lifetime, a value representing a fluorescence lifetime, an emission spectrum, an excitation fingerprint, and a fluorescence anisotropy of the fluorescent dyes.

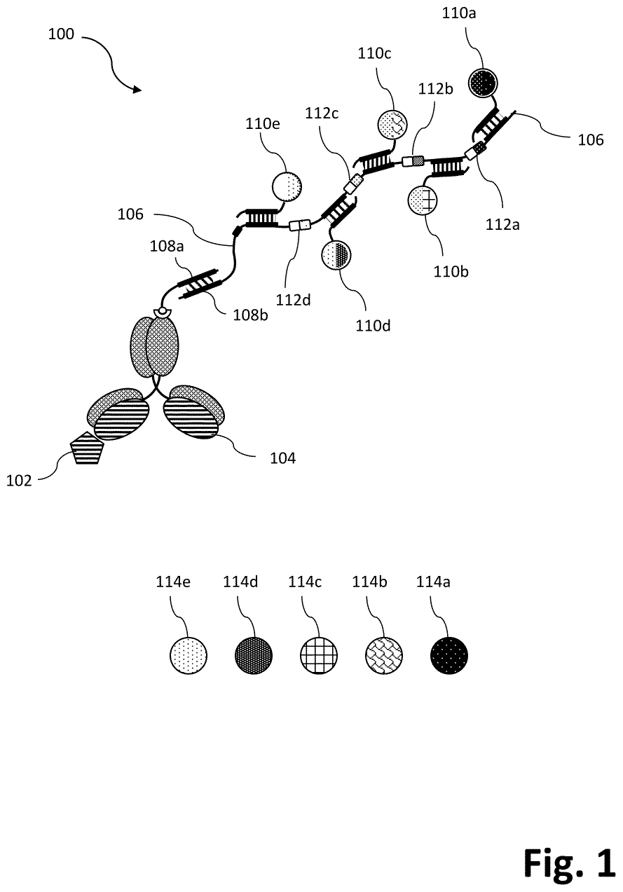

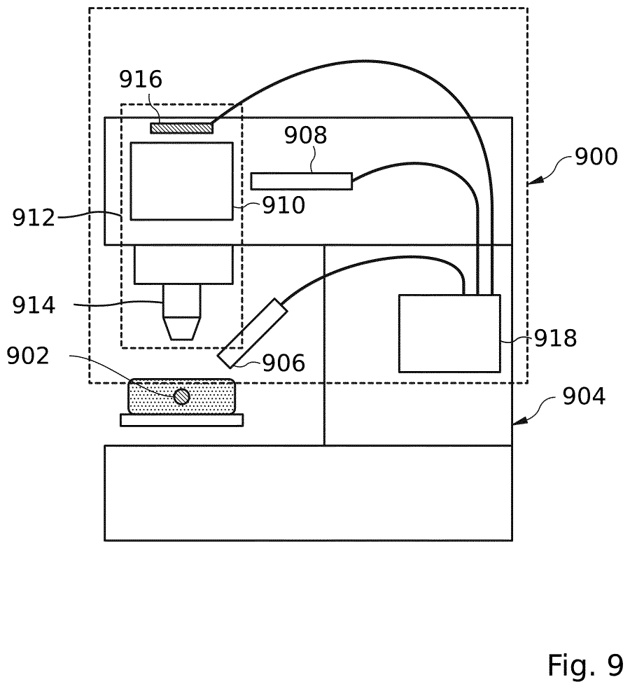

FIG. 1 is a schematic view of a marker 100 for marking a predetermined structure 102 within a biological sample 902 (c.f. FIG. 9) according to an embodiment.

The predetermined structure 102 may be a specific bio-molecule or a single analyte, for example a metal ion, located within a sample. In FIG. 1 the predetermined structure 102 is shown as a pentagon.

The marker 100 comprises an affinity reagent 104 and a linker structure 106 connected to the affinity reagent 104. The affinity reagent 104 is configured to attach itself to the predetermined structure 102, thereby connecting the linker structure 106 to the predetermined structure 102. A cross-linking agent, for example glutaraldehyde, may be used to strengthen the bond between the affinity reagent 104 and the predetermined structure 102. The affinity reagent 104 comprises an attachment site 108a configured to connect to a complementary attachment site 108b of the linker structure 106, thereby connected the linker structure 106 to the affinity reagent 104. The attachment site 108a and the complementary attachment site 108b are unique to the marker 100 and may for example be formed by oligomers such as an oligonucleotide comprising a unique sequence of nucleotides.

The linker structure 106 is exemplary formed as long chain along which five fluorescent dyes 110a, 110b, 110c, 110d, 110e are arranged. In other words: In the present embodiment, the fluorescent dyes 110a, 110b, 110c, 110d, 110e are arranged linearly along the linker structure 106. This linear arrangement of the fluorescent dyes 110a, 110b, 110c, 110d, 110e is schematically shown in a lower part of FIG. 1 as a sequence of five circles 114a, 114b, 114c, 114d, 114e, each circle 114a, 114b, 114c, 114d, 114e representing one of the fluorescent dyes 110a, 110b, 110c, 110d, 110e. A filling of the circles 114a, 114b, 114c, 114d, 114e corresponds to an emission characteristic of the respective fluorescent dye 110a, 110b, 110c, 110d, 110e, for example an emission spectrum, an emission lifetime or an emission fingerprint.

In the present embodiment, the fluorescent dyes 110a, 110b, 110c, 110d, 110e are indirectly bound to the linker structure 106. The fluorescent dyes 110a, 110b, 110c, 110d, 110e are connected to oligonucleotide sequences that are attached to complementary oligonucleotide sequences arranged on the linker structure 106. However, the fluorescent dyes 110a, 110b, 110c, 110d, 110e may also be directly, i.e. covalently bound to the linker structure 106. The combination of fluorescent dyes 110a, 110b, 110c, 110d, 110e is unique to the marker 100. This allows the marker 100 to be uniquely identified. By using a combination of fluorescent dyes 110a, 110b, 110c, 110d, 110e instead of a single dye, the number of markers 100 that can be uniquely identified is vastly increased. For example, 10 different fluorescent dyes are used. There are 45 unique two-dye combinations and 252 unique five-dye combinations. This means, that up to 252 different predetermined structures 102 can be marked and uniquely identified with only 10 fluorescent dyes.

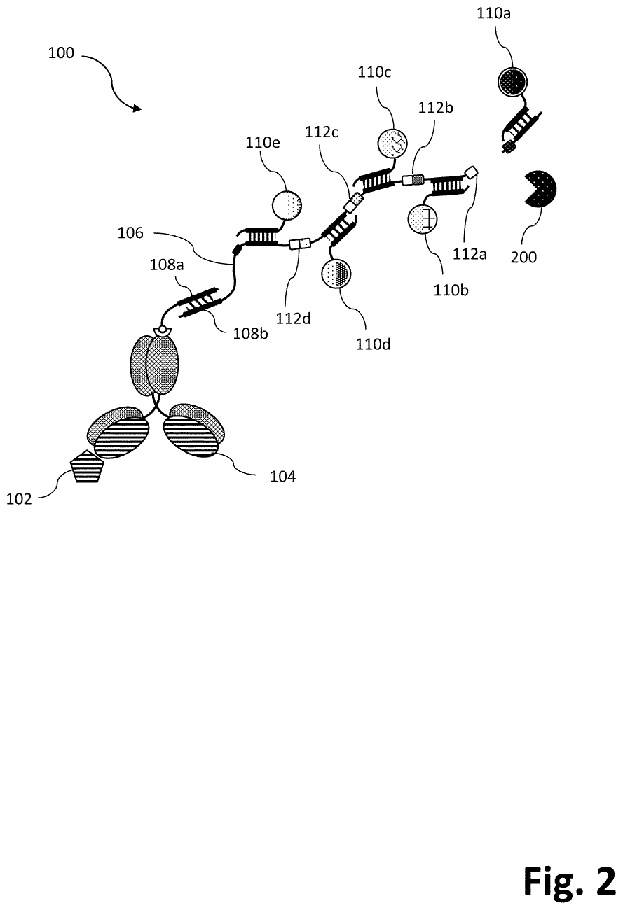

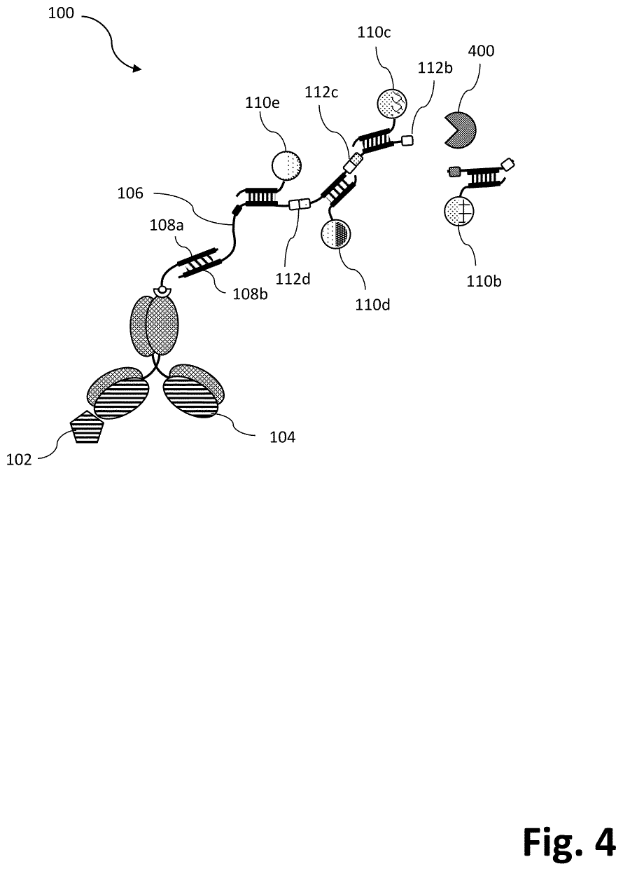

Cleavage sites 112a, 112b, 112c, 112d are arranged between the three complementary oligonucleotide sequences to which the fluorescent dyes 110a, 110b, 110c, 110d, 110e are connected. The linker structure 106 can be cut at the cleavage sites 112a, 112b, 112c, 112d by cleaving agents 200, 400 (c.f. FIGS. 2 and 4) in order to remove one of the fluorescent dyes 110a, 110b, 110c, 110d, 110e from the linker structure 106. When a first cleavage site 112a is cut by a first cleaving agent 200, a first fluorescent dye 110a is removed. When a second cleavage site 112b is cut by a second cleaving agent 400, a second fluorescent dye 110b is removed and only a third, fourth, and fifth fluorescent dye 110e, 110d, 110e remain attached to the marker 100. The cutting of the linker structure 106 is described in more detail below with reference to FIGS. 2 and 4.

FIG. 2 is a schematic view of the marker 100 according to FIG. 1 after the introduction of the first cleaving agent 200.

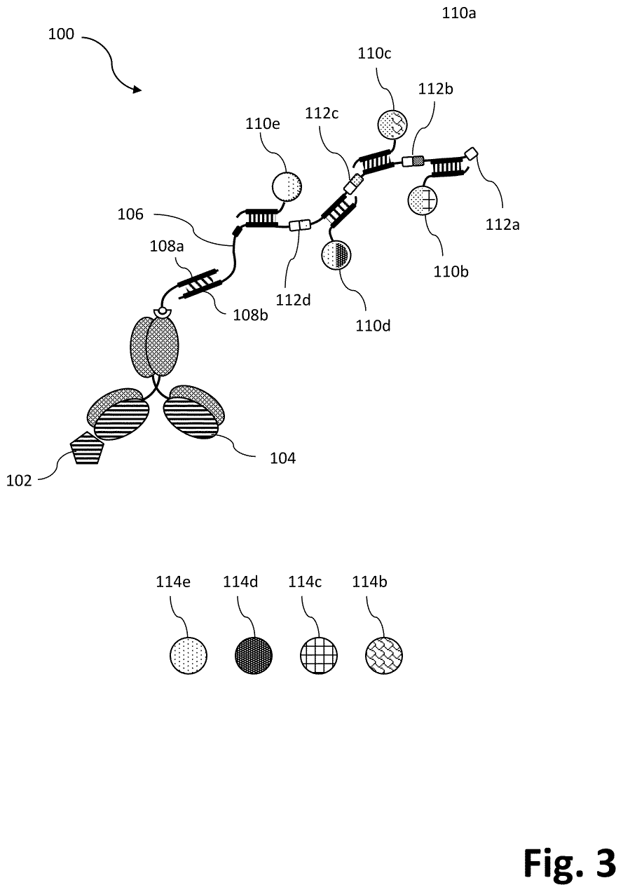

In the present embodiment, the cleaving agents 200, 400 are enzymatic cleaving agents. Alternatively, the cleaving agents 200, 400 may also be light, in particular UV-light, that cuts the linker structure 106 at the cleavage sites 112a, 112b, 112c, 112d by means of photolysis. In FIG. 2, the linker structure 106 is cut between a first and second fluorescent dyes 110a, 110b by the first cleaving agent 200. Thereby, the first fluorescent dye 110a removed from the marker 100. The removed fluorescent dye 110a may then be washed out of the sample 902. By removing the fluorescent dye 110a, the arrangement of fluorescent dyes 110a, 110b, 110c, 110d, 110e is changed. This is described below with reference to FIG. 3.

FIG. 3 is a schematic view of the marker 100 according to FIGS. 1 and 2 after the removal of the first fluorescent dye 110a.

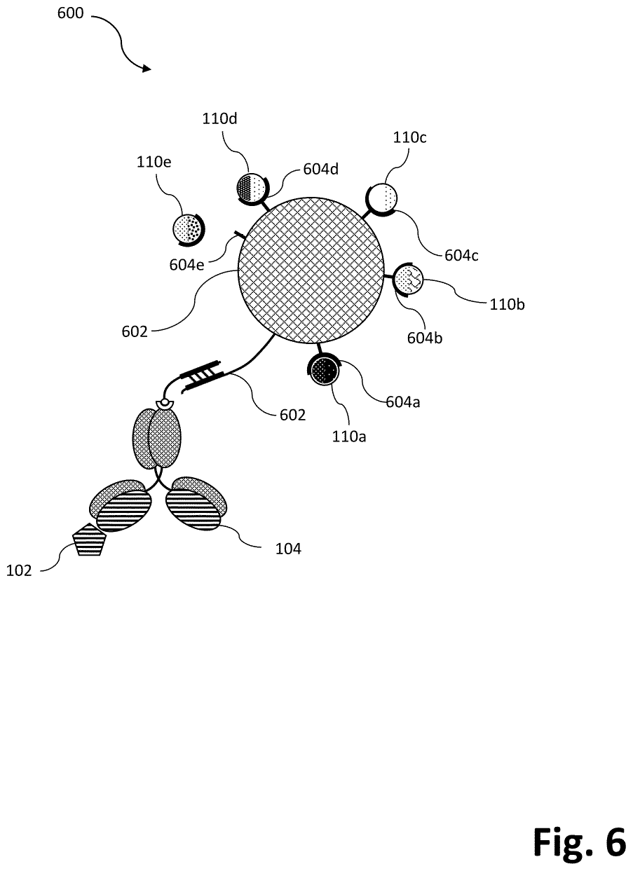

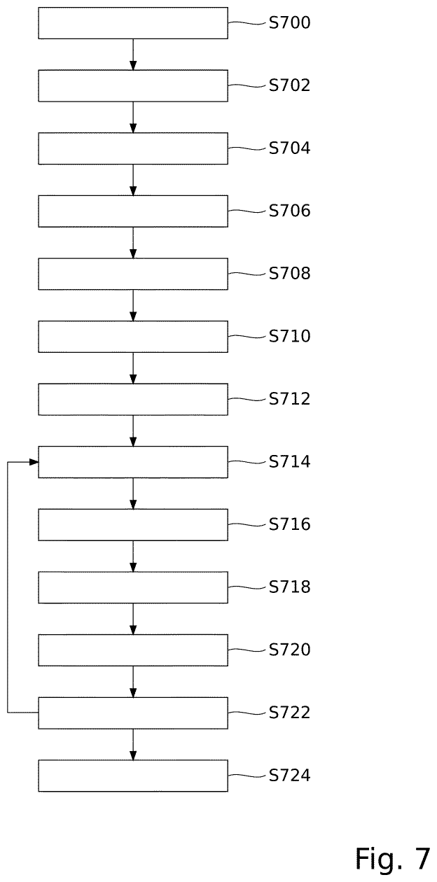

After the first fluorescent dye 110a is removed from the marker 100, four fluorescent dyes 110b, 110c, 110d, 110e remain attached to the marker 100. This new arrangement of the fluorescent dyes 110b, 110c, 110d, 110e is schematically shown in a lower part FIG. 3 as a sequence of four circles 114b, 114c, 114d, 114e, each circle representing one of the remaining fluorescent dyes 110b, 110c, 110d, 110e. By changing the arrangement, the code represented by the arrangement has also changed. This can be used to resolve ambiguities as is described below with reference to FIGS. 6 and 7.

FIG. 4 is a schematic view of the marker 100 according to FIGS. 1 to 3 after the introduction of the second cleaving agent 400.

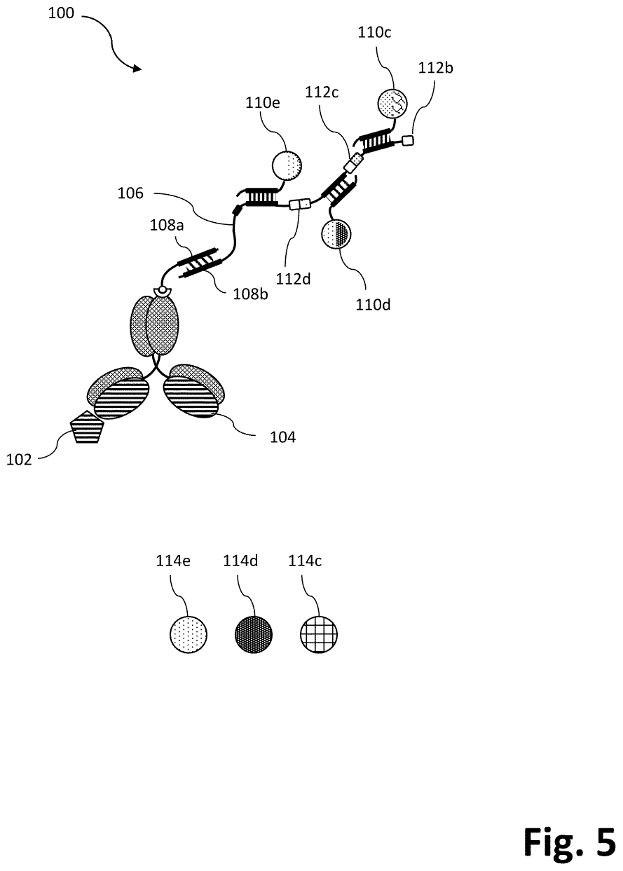

In FIG. 4, the linker structure 106 is cut between the second and a third fluorescent dye 110b, 110c by the second cleaving agent 400. Thereby, the second fluorescent dye 110b removed from the marker 100. The removed fluorescent dye 110b may then be washed out of the sample 902. By removing the fluorescent dye 110b, the arrangement of fluorescent dyes 110c, 110d, 110e is changed again, as is described below with reference to FIG. 5.

FIG. 5 is a schematic view of the marker 100 according to FIGS. 1 to 5 the removal of the first and second fluorescent dyes 110a, 110b.

After the second fluorescent dye 110b is also removed from the marker 100, only three fluorescent dyes 110c, 110d, 110e remain attached to the marker 100. This new arrangement of the fluorescent dyes 110c, 110d, 110e is schematically shown in a lower part FIG. 5 as a sequence of three circles 114c, 114d, 1142e, each circle representing one of the remaining fluorescent dyes 110c, 110d, 110e.

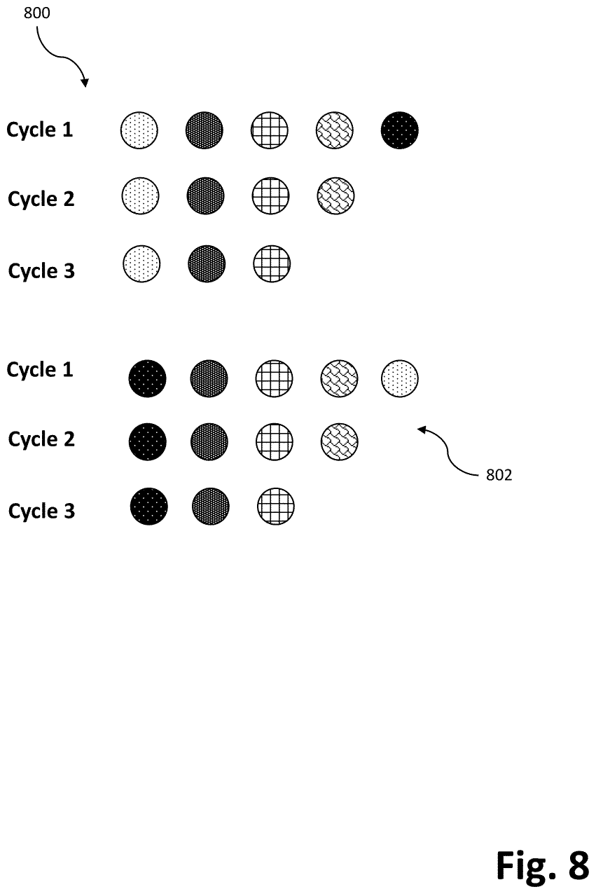

FIGS. 1 to 5 illustrate a method using the marker 100 for analyzing the biological sample 902 in three rounds or cycles of imaging. After each round or cycle one of the fluorescent dyes 110a, 110b, 110c, 110d, 110e is removed from the marker 100, thereby changing the arrangement of fluorescent dyes 110a, 110b, 110c, 110d, 110e. The method is described below in more detail with reference to FIGS. 7 and 8. The method may be performed with a device that is described below with reference to FIG. 9.

FIG. 6 is a schematic view of the marker 100 according another embodiment.

The marker 600 according the FIG. 6 is distinguished from the marker 100 according to FIGS. 1 to 5 in the shape of its linker structure 602. The linker structure 602 of the marker 600 according to the present embodiment may have a spherical geometry like for example when a polystyrene bead is used as support, a rod-like, sheet-like/planar, a polyedrical or other complex geometry when for example DNA-origami-based structures such as nanorulers are used as part of the linker structure 602. The fluorescent dyes 110a, 110b, 110c, 110d, 110e of the marker 600 are arranged in a star-like fashion around the linker structure 602. In the present embodiment, the cleavage sites 604a, 604b, 604c, 604d, 604e are arranged between the linker structure 602 and the fluorescent dyes 110a, 110b, 110c, 110d, 110e of the marker 600.

FIG. 7 is a flowchart of the method for analyzing the biological sample 902 utilizing the marker 100 described above with reference to FIGS. 1 to 6.

The process is started in step S700. In step S702 at least one marker 100 as described above is provided. The markers 100, 600 may for example be provided in a solution or a lyophilized solid. In particular, the affinity reagent 104 and different linker structure 106s are provided separately.

In step S704 the affinity reagents 104 are introduced into the sample 902. The linker structures 106, 602 may already be attached to the affinity reagents 104 when the markers 100, 600 are introduced into the sample 902. Alternatively, the affinity reagent 104 and the linker structures 106, 602 are introduced separately into sample 902. After the affinity reagents 104 had time to attach themselves to their respective predetermined structures 102 and linker structures 106, 602 had time to attach themselves to their respective affinity reagents 104, unbound affinity reagents 104 and linker structures 106, 602 are washed out of the sample 902 in an optional step S706. It may be the case that a specific predetermined structure 102 a marker 100, 600 is supposed to attach itself to is not present in the sample 902. Washing out unbound markers 100, 600 thus ensures that only markers 100, 600 that are actually attached to their associated predetermined structure 102 are detected in the following. It may also be the case that not every linker structure 106 binds itself to its associated affinity reagent 104. In both cases, washing out unbound markers 100, 600 and linker structures 106, 602 prevents misidentification of structures in the sample 902.

In step S708, at least one first excitation light is directed onto the sample 902 in order to excite the fluorescent dyes 110a, 110b, 110c, 110d, 110e of the marker 100, 600. The first excitation light may comprise light of a single wavelength or wavelength spectrum depending on the specific fluorescent dyes 110a, 110b, 110c, 110d, 110e. More than one first excitation light may be used, for example light emitted by different light sources either simultaneously or sequentially. The excited fluorescent dyes 110a, 110b, 110c, 110d, 110e then emit fluorescence light which is used to generate the at least one first readout in step S710. The first readout comprises information about the fluorescence light, in particular an emission spectrum, a fluorescence emission intensity, a fluorescence lifetime or an excitation fingerprint of the fluorescent dyes 110a, 110b, 110c, 110d, 110e. The information from the first readout is used to identify the fluorescent dyes 110a, 110b, 110c, 110d, 110e, and thus the markers 100, 600 present in the readout volume of the sample 902 in step S712. The steps S704 to S712 correspond to a first cycle or round of imaging the sample 902.

Optionally, if every marker 100, 600 in the sample 902 was identified with at least a predetermined certainty from the first readout alone, the process may be ended after step S712. Alternatively, the process is continued in step S714.

In step S714 the first cleaving agent 200 is introduced into the sample 902. After the linker structure 106 has been cut such that the first fluorescent dyes 110a are removed, the removed fluorescent dyes 110a are washed out of the sample 902 in step S716. This changes, which fluorescent dyes 110a, 110b, 110c, 110d, 110e are connected to the markers 100, 600. How this can be used to resolve ambiguities and to increase the number of markers 100, 600 that can be used in a single experiment is described below with reference to FIG. 8. In step S718 second excitation light is directed onto the sample 902 in order to excite the remaining fluorescent dyes 110b, 110c, 110d, 110e of the marker 100, 600. The second excitation light may also comprise light of a single wavelength or wavelength spectrum depending on the specific fluorescent dyes 110b, 110c, 110d, 110e remaining in the sample 902. Likewise, more than one second excitation light may be used. In step S720 at least one second readout is generated from the fluorescence light emitted by the excited fluorescent dyes 110a, 110b, 110c, 110d, 110e located in the readout volume of the sample 902. The information from the second readout is used to identify the fluorescent dyes 110a, 110b, 110c, 110d, 110e of the markers 100, 600 and thus the markers 100, 600 present in the sample 902 in step S722. The steps S714 to S722 correspond to a second cycle or round of imaging the sample 902.

If every marker 100, 600 in the sample 902 was identified with at least a predetermined certainty from the first and second readouts, the process is ended in step S724. Alternatively, the steps S714 to S722 are repeated in a third cycle or round of imaging the sample 902. In the third circle, the second cleaving agent 400 is used instead of the first cleaving agent. In subsequent cycles or rounds, yet another cleaving agent may be used.

FIG. 8 shows a schematic drawing of the arrangements of the fluorescent dyes of two different markers.

An upper view 800 of FIG. 8 shows the arrangement of the fluorescent dyes of a first marker. A lower view 802 of FIG. 8 shows the arrangement of the fluorescent dyes of a second marker. Each of the fluorescent dyes is represented in FIG. 8 by a circle. A filling of each circle corresponds to an emission characteristic of the respective fluorescent dye, for example an emission spectrum, an emission lifetime or an emission fingerprint. The arrangement of the fluorescent dyes of the two markers is shown for three cycles each of the method described above with reference to FIG. 8.

As can be seen in FIG. 8, the two markers comprise the same fluorescent dyes. This means, although the arrangements of the fluorescent dyes are different for each markers, the markers cannot be differentiated based on the emission characteristics of their respective fluorescent dyes alone. In order to resolve this ambiguity, one of the fluorescent dyes of each marker is removed after each cycle. The changes, which fluorescent dyes are attached to which marker. A first fluorescent dye of the first has the same emission characteristics as a fifth fluorescent dye of the second marker. Likewise, a fifth fluorescent dye of the first has the same emission characteristics as a first fluorescent dye of the second marker. By removing the first fluorescent dye of each of the markers, the markers are now different by one fluorescent dye. Thus, the markers can be differentiated starting with the second cycle.

FIGS. 1 to 8 describe a marker 100, 600 and a method that can be used to increase the overall number of markers 100, 600 that can be reliably differentiated in a single experiment, and thus increasing the number of structures inside the sample 902 that can be identified. Going back to the example described with reference to FIG. 1: If 10 different fluorescent dyes are used, and each markers 100, 600 comprises 5 different fluorescent dyes, there are 252 unique combination of fluorescent dyes. However, the method described above is sensitive to the arrangement of the fluorescent dyes, so to speak. This further increases the number of unique combination of fluorescent dyes by a factor 5!, meaning that over 30000 unique combination of fluorescent dyes are possible with 10 different fluorescent dyes alone.

FIG. 9 shows a schematic drawing of a device 900 for analyzing a biological sample 902.

In particular, the device 900 is capable of performing the method for analyzing a biological sample 902 described above with reference to FIGS. 6 to 7 utilizing markers 100, 600 described above with reference to FIGS. 1 to 5. In Figure the device 900 is exemplary shown as being a part of a microscope system 904.

The device 900 comprises a staining unit 906 for introducing the marker 100, 600 into the sample 902. For that purpose, the staining unit 906 may comprise one or more pipettes that may or may not be automated. The device 900 also comprises an excitation unit 908 for exciting the fluorescent dyes 110a, 110b, 110c, 110d, 110e of the markers 100, 600. The excitation unit 908 comprises at least one light source, preferably a coherent light source. The at least one light source is configured to emit the excitation lights used for exciting the fluorescent dyes 110a, 110b, 110c, 110d, 110e. In order to emit excitation light of different wavelengths or wavelength spectra, the light source may be a tunable light source. Alternatively, the device 900 may comprise two or more light sources with emitting light of different wavelengths or wavelength spectra. In the embodiment shown in FIG. 9, the excitation lights emitted by the excitation unit 908 is directed onto the sample 902 by a beam splitting unit 910.

An imaging unit 912 of the device 900 is configured to generate images from the fluorescence light emitted by the excited fluorescent dyes 110a, 110b, 110c, 110d, 110e. The images being the readouts in this embodiment. The imaging unit comprises an objective 914 directed at the sample 902 for capturing the fluorescence light. The captured fluorescence light is then directed onto a detection unit 916 by the beam splitting unit 910. The detection unit 916 comprises at least one detector element and a diffractive element for splitting the fluorescence light into different detection channels.

After generating a readout, at least one of the fluorescent dyes 110a, 110b, 110c, 110d, 110e needs to be removed from its respective marker 100, 600 and the sample 902. This is done by means of the cleaving agents 200, 400 200, 400. The cleaving agents 200, 400 200, 400 may be in particular be enzymatic cleaving agents 200, 400, which can be introduced into the sample 902 by means of the staining unit 906. Alternatively, the light source of the excitation unit 908 or an additional light source may be used to cut the linker structures 106, 602 by means of photolysis.

The device 900 further comprises a processor 918 connected to the staining unit 906, the excitation unit 908 and the detection unit 916. The processor 918 is configured to control the elements of the device 900 in order to perform the method for analyzing a biological sample 902. In particular, the processor 918 is configured to perform the method based on at least one user input.

Identical or similarly acting elements are designated with the same reference signs in all Figures. As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SIGNS

100 Marker
102 Predetermined structure
104 Affinity reagent
106 Linker structure
108a Attachment site
108b Complementary attachment site
110a, 110b, 110c, 110d, 110e Fluorescent dye
112a, 112b, 112c, 112d Cleavage site
114a, 114b, 114c, 114d, 114e Circle
200 Cleaving agent
400 Cleaving agent
600 Marker
602 Linker structure
604a, 604b, 604c, 604d, 604e Cleavage site
900 Device
902 Sample
904 Microscope system
906 Staining unit
908 Excitation unit
910 Beam splitting unit
912 Imaging unit
914 Objective
916 Detection unit
918 Processor

1: A marker for marking a predetermined structure within a biological sample, the marker comprising:
an affinity reagent configured to attach to the predetermined structure,
a linker structure attached to the affinity reagent and extending from the affinity reagent, and
at least two different fluorescent dyes arranged at the linker structure,
wherein the linker structure comprises at least one cleavage site arranged between the two fluorescent dyes or between one of the fluorescent dyes and the linker structure, and
wherein the linker structure is capable of being cut at the cleavage site by a cleaving agent in order to remove at least one of the fluorescent dyes from the marker.

2: The marker according to claim 1, wherein the affinity reagent comprises an attachment site, and wherein the linker structure comprises a complementary attachment site configured to attach to the attachment site of the affinity reagent.

3: The marker according to claim 1, wherein the linker structure is formed by oligonucleotides or peptides.

4: The marker according to claim 1, wherein the cleavage site is an enzymatic cleavage site, and the linker structure is capable of being cut at the cleavage site by an enzymatic cleaving agent.

5: The marker according to claim 4, wherein the enzymatic cleavage site is a target site of a restriction enzyme, a CRISPR/Cas target, or a recombinase target site.

6: The marker according to claim 1, wherein the cleavage site is a photocleavage site, and the linker structure is capable of being cut at the cleavage site by photolysis.

7: The marker according to claim 1, wherein the cleavage site is a proteolytic cleavage site, and the linker structure is capable of being cut at the cleavage site by a protease.

8: A method for analyzing a sample, the method comprising:
providing at least two markers according to claim 1, wherein the arrangement of the fluorescent dyes or the cleavage site at the linker structure is unique for each of the markers;
introducing the markers into the sample;
directing at least one first excitation light onto the sample in order to excite fluorescent dyes of the markers;
generating at least one first readout from fluorescence light emitted by the excited fluorescent dyes located in a readout volume of the sample;
introducing at least one cleaving agent into the sample in order to remove at least one of the fluorescent dyes from a respective marker;
directing at least one second excitation light onto the sample in order to excite remaining fluorescent dyes of the markers;
generating at least one second readout from fluorescence light emitted by the excited remaining fluorescent dyes located in the readout volume of the sample; and
determining the markers present in the readout volume based on the first readout and second readout.

9: The method according to claim 8, wherein generating the first readout or the second readout comprises separating the fluorescence light emitted by the excited fluorescent dyes or by the excited remaining fluorescent dyes into detection channels,
wherein the detection channels correspond to at least one emission characteristic and/or excitation characteristic of the fluorescent dyes, and
wherein the emission characteristic is one of: an emission spectrum, a fluorescence intensity, a fluorescence lifetime, or an excitation fingerprint.

10: The method according to claim 9, wherein each marker is configured such that each fluorescent dye corresponds to one detection channel of the first readout or the second readout, respectively.

11: The method according to claim 8, wherein the fluorescent dyes of the markers are divided into sets of fluorescent dyes;
wherein the fluorescent dyes in a same set are capable of being excited by a same wavelength or by a same wavelength spectrum;
wherein at least one of the first excitation light and the second excitation light is directed at the sample in order to excite the fluorescent dyes of the respective set;
wherein at least one of the first readout and the second readout is generated from the fluorescence light emitted by the respective set of fluorescent dyes located in the readout volume of the sample.

12: The method according to claim 11, wherein the first excitation light and the second excitation light are directed onto the sample in a sequence temporally following each other.

13: The method according to claim 8, wherein the at least one removed fluorescent dye is washed out of the sample before the second excitation light is directed onto the sample.

14: The method according to claim 8, wherein the first readout and/or the second readout comprises at least one image of the readout volume, or a readout signal data stream of the readout volume, or a readout image data stream of the readout volume.

15: The method according to claim 8, further comprising capturing a hyperspectral image of the sample in order to generate the first readout and/or the second readout.

16: The method according to claim 8, further comprising stabilizing a fluorescence lifetime of at least one fluorescent dye, by placing the at least one fluorescent dye in a shielded environment by at least one of encapsulating, polymer-matrix embedding, co-crystallizing, or binding to a DNA origami nanostructure with a hollow core.

17: A device for analyzing a biological sample being adapted to carry out the method according to claim 8.

18: The device according to claim 17, comprising a microscope, a plate reader, a cytometer, an imaging cytometer, or a fluorescence activated cell sorter configured to generate the first readout and the second readout.

19: The device according to claim 17, configured to determine at least one of: a fluorescence emission intensity, a fluorescence lifetime, a value representing a fluorescence lifetime, an emission spectrum, an excitation fingerprint, or a fluorescence anisotropy of the fluorescent dyes.
